(19) United States
(12) Patent Application Publication
(10) Pub. No.: US 2018/0110748 A1
(43) Pub. Date: Apr. 26, 2018

(54) COMPOSITION FOR REDUCING OR SUPPRESSING INCREASE IN NEUTRAL FAT LEVEL CONTAINING N-3 UNSATURATED FATTY ACID, AND USE OF N-3 UNSATURATED FATTY ACID IN PRODUCTION OF SAME COMPOSITION

(71) Applicants: MARUHA NICHIRO CORPORATION, Tokyo (JP); National Institute of Advanced Industrial Science and Technology, Tokyo (JP)

(72) Inventors: Tatsuya Konishi, Ibaraki (JP); Yasuhiko Shiina, Ibaraki (JP); Yoshinori Takahashi, Ibaraki (JP); Katsutaka Oishi, Ibaraki (JP); Chiaki Hasimoto, Ibaraki (JP); Saori Yamamoto, Ibaraki (JP)

(73) Assignees: MARUHA NICHIRO CORPORATION, Tokyo (JP); National Institute of Advanced Industrial Science and Technology, Tokyo (JP)

(21) Appl. No.: 15/789,738

(22) Filed: Oct. 20, 2017

(30) Foreign Application Priority Data
Oct. 21, 2016 (JP) 2016-207252
Apr. 25, 2017 (JP) 2017-086453
Jul. 31, 2017 (JP) 2017-148342

(51) Int. Cl. A61K 31/202; A61K 35/60; A61K 9/00; A23L 33/12

(52) U.S. Cl. CPC A61K 31/202; A61K 35/60; A23V 2002/00; A23L 33/12; A61K 9/0056

(57) ABSTRACT

A composition which can efficiently obtain an effect of decreasing or suppressing an increase in the neutral fat level is provided.

Docosahexaenoic acid (DHA) and/or eicosapentaenoic acid (EPA) is used as an active ingredient of a composition for decreasing or suppressing an increase in the neutral fat level, and the timing of intake of the active ingredient is within a time for intake within 6 hours after arousal from sleep including the breakfast time essentially including the breakfast time.

### BACKGROUND OF THE INVENTION

### 1. Field of the Invention

The present invention relates to a composition for reducing or suppressing an increase in the neutral fat level containing, as an active ingredient, docosahexaenoic acid and/or eicosapentaenoic acid as an n-3 unsaturated fatty acid, and a method of using docosahexaenoic acid and/or eicosapentaenoic acid in production of the composition.

Further, the present invention relates to a method of treating a subject with a composition according to the present invention, which makes it possible to reduce neutral fats by increasing the concentration of n-3 unsaturated fatty acids (DHA.EPA) in blood and decreasing the concentration of n-6 unsaturated fatty acids in human blood to enhance the n-3/n-6 ratio by intake of the composition comprising a fish oil within 6 hours after arousal from sleep including the first meal (breakfast).

Further, the present invention related to use of the composition for reducing or suppressing an increase in the neutral fat level of a subject.

### 2. Description of the Related Art

Docosahexaenoic acid (DHA) and eicosapentaenoic acid (EPA) are highly unsaturated fatty acids classified into the n-3 (ω-3) fatty acid, and their contents are known to be high in the fish oils obtained from fishes such as herring, mackerel, sardine, tuna, bonito, saury, adult yellowtail, etc.

It has been reported that DHA had various physiologically active functions such as a platelet aggregation suppressing action, a blood neutral fat level reducing action, a blood cholesterol level deducing action, a brain function improving effect, etc.

It has been reported that also EPA had various physiologically active functions such as a platelet aggregation suppressing action, a blood neutral fat level reducing action, a blood cholesterol level reducing action, etc.

DHA and EPA have been used as an active ingredient of supplements and medicaments for reducing the neutral fat level.

Non-Patent Document 1 discloses that a fish oil has an effect of reducing the plasma neutral fat level. Further, Patent Document 1 discloses a composition containing a fish oil for controlling fat metabolism.

Patent Document 2 discloses that DHA has an activity for reducing the blood neutral fat level.

Non-Patent Document 2 is a report concerning in vivo kinetics of ethyl eicosapentaenoate (EPA-E), and discloses that EPA has an activity for reducing neutral fats in blood.

On the other hand, the chronobiology for evaluating biological rhythm and circadian rhythm has been established recently. A correlation between timing of intake of a drug and the effect thereof and a correlation between timing of intake of meals or nourishments and the effect thereof have been studied.

Non-Patent Document 3 describes that timing of meal intake is important for suitable body weight maintenance and for prophylaxis of onset of metabolic syndrome, as well as that the influence to sugar-fat metabolism and the effects for suppressing obesity by DHA/EPA has been verified by their intake in the morning and the evening without effecting fasting.

Patent Document 3 discloses that an increase in the blood glucose level after a meal could be suppressed by orally administering DHA at a time between meals within from 2 to 6 hours after a meal and until 1 hour before the next meal.

### PRIOR ART DOCUMENT

### Patent Document

[Patent Document 1] WO2009/050580

[Patent Document 2] Japanese Patent Laid-Open No. H08-140636; JP, 08-140636, A

[Patent Document 3] WO2012/063820

[Non-Patent Document 1] Biochimica et Biophysics Acta, 792, (1984), 103-109

[Non-Patent Document 2] J. Lipid Nutr., Vol. 24, No. 1, (2015), 21-32

[Non-Patent Document 3] Food and Development. Vol. 51, No. 1, UBM Media Co., Ltd., published on Jan. 1, 2016, pp. 4 to 6

### SUMMARY OF THE INVENTION

An object of the present invention is to provide a composition and its use for reducing or suppressing an increase in the neutral fat level, which comprises, as an active ingredient, DHA and/or EPA, which can efficiently provide the effect of reducing or suppressing an increase in the neutral fat level by DHA and/or EPA.

An embodiment of the present invention relates to a composition for reducing or suppressing an increase in a neutral fat level according to the present invention, which is characterized in containing docosahexaenoic acid (DHA) and/or eicosapentaenoic acid (EPA), as an active ingredient, for intake by a subject in need of intake of the composition within a time range for intake essentially including a breakfast time, and the time range for intake is defined as the time range within 6 hours after arousal from sleep essentially including the breakfast time.

According to the present invention, “the first meal” within 6 hours after arousal from sleep is defined as “breakfast”.

An embodiment of the present invention relates to use of a composition for decreasing or suppressing an increase in a neutral fat level of a subject in need of intake of the composition,

wherein the composition contains docosahexaenoic acid (DHA) and/or eicosapentaenoic acid (EPA) as an active ingredient; and

wherein the composition is taken by the subject in a time range for intake, essentially including a breakfast time and within 6 hours after arousal from sleep including the breakfast time.

An embodiment of the present invention relates to a method of treating a subject in need of treatment for decreasing or suppressing an increase in the neutral fat level comprising:

providing the subject a composition for the treatment,

wherein the composition contains docosahexaenoic acid (DHA) and/or eicosapentaenoic acid (EPA) as an active ingredient; and

wherein the composition is taken by the subject in a time range for intake, essentially including a breakfast time and within 6 hours after arousal from sleep including the breakfast time.

An embodiment of the present invention relates to a method of using an active ingredient for decreasing or suppressing an increase in the neutral fat level, in production of a composition for decreasing or suppressing an increase in the neutral fat level,

wherein the active ingredient comprises docosahexaenoic acid (DHA) and/or eicosapentaenoic acid; and

wherein the composition is provided for intake by a subject in a time range for intake, essentially including a breakfast time and within 6 hours after arousal from sleep including the breakfast time.

As the above-described active ingredient, a fish oil can be used.

The above-described composition can be in the forms of pharmaceutical products or food products including functional food products.

According to the composition for reducing or suppressing an increase in a neutral fat level according to the present invention, the effect of reducing or suppressing an increase in the neutral fat level can be efficiently obtained without exerting an influence on the blood glucose level, by setting the intact timing within 6 hours after arousal from sleep essentially including the breakfast time.

### BRIEF DESCRIPTION OF THE DRAWINGS

FIG. 1 is a drawing illustrating an experiment protocol concerning a time-limited feeding of a fish oil-containing feed.

FIG. 2 shows graphs relating to the concentrations of glucose, free fatty acid, neutral fat and total cholesterol measured in the plasma, which was obtained by killing mice every 6 hours from 2:00 o'clock after fish oil time-limited feeding for 2 weeks under the light-dark cycles consisting of 12 hours of the light period and 12 hours of the dark period (lighting at 0:00 o'clock, lights-out at 12:00 o'clock), and separating the plasma from the blood of each mouse.

FIG. 3 shows graphs relating to the contents of free fatty acid, neutral fat and total cholesterol per weight of liver tissue.

FIG. 4 shows graphs relating to the analysis results of the expression amounts of fatty acid synthesis related genes (Fasn, Acc1, Scd1).

FIG. 5 shows graphs relating to the fatty acid concentration measured in the plasma obtained by killing mice every 6 hours from 2:00 o'clock after fish oil time-limited feeding for 2 weeks, and separating the plasm from the blood from each mouse.

FIG. 6 shows graphs relating to the amounts of fatty acid and n-3 unsaturated fatty acid and the n-3/n-6 ratio measured in the plasma, which was obtained by killing mice at 0, 6, 10, 14 and 18 hours after administration of a fish oil single dose at 1:00 o'clock and 13:00 o'clock, and separating the plasm from the blood from each mouse.

FIG. 7 shows graphs relating to the amounts of fatty acid amount and n-3 unsaturated fatty acid and the n-3/n-6 ratio measured in the plasma, in Example 4.

FIG. 8 is a graph showing the transition of blood neutral fat in Example 5.

FIG. 9 is a graph showing the transition of blood n-6 unsaturated fatty acid in Example 5.

FIG. 10 is a graph showing the transition of blood n-3 unsaturated fatty acid in Example 5.

FIG. 11 is a graph showing the transition of blood n-3/n-6 ratio in Example 5.

FIG. 12 is a graph showing the transition of blood saturated fatty acid in Example 5.

### DESCRIPTION OF THE PREFERRED EMBODIMENTS

It is known that DHA and EPA have various physiologically active functions. The present inventors have focused on the effect of reducing or suppressing an increase in a neutral fat level and, thus, intensively studied the form of a composition containing them as an active ingredient and the timing for intake or administration thereof to more efficiently provide the effect.

In experiments of mice conducted using fish oils containing DHA and EPA, an increase in the blood DHA and EPA concentrations and an effect of reducing the neutral fat level were recognized, throughout the day, in the mice by uptake of the fish oils in the activation beginning early stage of a day including a time of arousal from sleep in the daily biological rhythm. The uptake of the fish oils did not exert any influence on the blood glucose level, the total cholesterol level and the blood free fatty acid level, throughout the day. These effects are considered to be obtained by an increase of incorporation of n-3 unsaturated fatty acids such as DHA, EPA, etc., into the blood by uptake of the fish oils in the activation beginning early stage of a day.

The mice as used by the present inventors in the experiments are nocturnal in habit, and usually have a daily biological rhythm in which the active phase including a time of arousal is included in 12 hours of the dark period, and they sleep in the light period.

The results of the experiments with the mice by the present inventors show that DHA and EPA contained in the fish oils act specifically on the neutral fat level depending on the intake timing of the fish oils, and such a fact is a novel finding by the present inventors.

Further, the human clinical trials were conducted and, thus, it was confirmed that the neutral fat level was lowered by reducing the concentration of n-6 unsaturated fatty acids to enhance the n-3/n-6 ratio by intake of a fish oil in the breakfast. Since also the blood saturated fatty acid concentration was lowered, it was also supported that it was possible that neutral fat resynthesis would be suppressed by acceleration of beta-oxidation.

The present invention has been completed based on the novel findings by the present inventors described above.

The composition according to the present invention contains DHA and/or EPA, as an active ingredient.

The present invention also relates to a method of using. DHA and/or EPA as an active ingredient for decreasing or suppressing an increase in the neutral fat level, in a production of a composition for decreasing or suppressing an increase in the neutral fat level.

The above-described composition is taken, in all cases, by a subject in need of its intake, in a period of timing for intake within a time range for intake essentially include a breakfast time, and the time range for intake is defined as the time range within 6 hours after arousal from sleep essentially including the breakfast time.

Further, the present invention relates to a method of reducing a neutral fat level or suppressing an increase in a neutral fat level of a subject in need of treatment using a composition, wherein the composition is taken in a period for intake defined as a time range for intake within 6 hours after arousal from sleep including the breakfast time, which essentially includes the breakfast time, and wherein the active ingredient comprises docosahexaenoic acid (DHA) and/or eicosapentaenoic acid (EPA).

The method of reducing a neutral fat level or suppressing an increase in a neutral fat level may comprise a step of feeding or administrating an effective amount of the composition including the active ingredient comprising DHA and/or EPA for a subject in need of intake of the composition in the specific time range for intake.

Further, the present invention relates to use of docosahexaenoic acid (DHA) and/or eicosapentaenoic acid (EPA) for reducing or suppressing an increase in a neutral fat level of a subject in need of treatment. wherein docosahexaenoic acid (DHA) and/or eicosapentaenoic acid (EPA) is taken in a period for intake defined as a time range for intake within 6 hours after arousal from sleep including the breakfast time, which essentially includes the breakfast time.

DHA and EPA can be used each singly or can be used in combination as an active ingredient. When both DHA and EPA are used as an active ingredient, fish oils can be suitably used.

As the fish oils, those containing DHA and EPA in various purities and compositions are commercially available or known, and those capable of achieving the object of the present invention can be selected from these fish oils.

Commercially available fish oils include, for example, DHA22 (purified tuna oil containing 22% DHA as the fatty acid composition), DHA22K (purified tuna oil and bonito oil containing 22% DHA as the fatty acid composition), DHA27W (purified tuna oil and bonito oil containing 27% DHA as the fatty acid composition), DHA46A (purified tuna oil containing 46% DHA as the fatty acid composition), DHA-RS (purified tuna oil and bonito oil containing 70% DHA as the fatty acid composition), etc. These DHA products are manufactured by Maruha Nichiro Corporation.

Regarding DHA and EPA in fish oils, the content of DHA is 5 to 70% by mass, which can be defined “by weight”, and the content of EPA is 01 to 45% by mass, while the ratio of DHA to EPA is 700:1 to 1:9 by mass, in general. It is possible to use fish oils having the contents of DHA and EPA in these ranges as an active ingredient of the composition according to the present invention.

The content of DHA and/or EPA in the composition according to the present invention is not particularly restricted providing that the intended effect of reducing or suppressing an increase in a neutral fat level can be obtained. The content of DHA and/or EPA may be selected from the range of 0.01 to 95% by mass, so as to provide an effective amount of the active ingredient in the composition.

The content of DHA and/or EPA in the composition according to the present invention may be selected according to at least one of the total intake amount (or total administration amount, i.e., total dose(s)) in the time range for intake and the form of the composition.

The total intake amount (or total feeding or administration amount, i. e., total dose(s)) in the time range for intake is not particularly restricted providing that the intended effect of reducing or suppressing an increase in a neutral fat level can be obtained. The total intake amount or the total dose(s) in the time range for intake can be selected from the range of 100 mg to 10000 mg, preferably 300 mg to 5000 mg, more preferably 500 mg to 2000 mg per day (body weight 60 kg).

It is important that DHA and/or EPA is taken by an organism in the early stage of an activity beginning period after arousal from sleep, i.e., the timing of intake of DHA and/or EPA in the daily biological rhythm, in order to obtain the effect intended in the present invention.

As the early stage of activity beginning period of an organism as the subject after arousal from sleep, the time range within 6 hours after arousal from sleep including the breakfast time, i. e., the breakfast time is essentially included, is used as a time for intake.

It is preferable that no lunch is included in the time range for intake.

In the above-described time range for intake, the composition according to the present invention can be taken by a single dose or multiple doses, essentially at the breakfast time and, if necessary, at the times other than the breakfast time within the time range for intake.

The timing of intake of the composition according to the present invention is preferably limited only in the above-described time range for intake, not only to suppress the consumption amount of DHA and/or EPT to the requisite minimum, but also to obtain the intended effect efficiently.

“Sleep” referred to in the present invention is a natural condition in which reaction to environmental stimuli is lowered and unconsciousness occurs, but arousal easy. When several such conditions are present in 24 hours of a day, only the condition continued for the longest time corresponds to “Sleep” referred to in the present invention. Thus, the sleep referred to in the present invention occurs only once in 24 hours of a day. For example, the so-called napping continuing for a relatively short period of time taken in daytime is different from the sleep as going to bet according to the present invention, which continues for several hours usually taken by a human in the night.

The sleeping time is not particularly restricted and is preferably 3 hours or more, further preferably 4 to 10 hours for the method of intake of a food product according to the present invention.

The period for conducting daily intake of a composition according to the present invention at the above-described specified timing may be set so as to obtain the effect of the present invention, and is not particularly restricted. It is preferable that the daily intake at the above-described specified timing is carried out continuously for a certain period. it is preferable that the daily intake at the above-described specified timing is carried out continuously for 2 weeks or more, particularly, 4 weeks or more, from the standpoint of more sufficient attainment of the effect according to the present invention.

The composition according to the present invention can be provided in various forms. For example. the composition according to the present invention can be provided in a form of a food product per se including a functional food; in a form of an additive used in production of food products including various processed food products and functional food products; in a form of an animal feed per se; in a form of an additive used in production of animal feeds; and in a form of a pharmaceutical formulation, etc. These various forms can be produced by the conventional methods usually used.

The food products as the subject, to which the present invention can be applyed, include the whole range of food products, which include beverages. The food products include the general processed food products including so-called health food products; food products with health claims and supplements such as special heath food and food with nutrient function with claims prescribed in the health-promoting food institute of Japanese Consumer Affairs Agency; the corresponding food products with health claims and supplements such as special heath food and food with nutrient function with claims in the countries other than Japan; and also feeds to be fed to animals.

The forms of a pharmaceutical formulation products includes oral liquid formulations, tablets, granules, powders, capsules, suppositories, eye-drops, jellies, etc. Also the food products such as functional food products, etc. can be provided in the forms of, for example, oral quid formulations, tablets, granules, powders, capsules, jellies, etc.

For preparation of the pharmaceutical formulation, various additives such as a carrier, an excipient, a diluent, a base material, etc. as used in drug manufacturing, can be used.

Various additives used for preparation include, for example, magnesium stearate, talc, lactose, dextrin, starches, methylcellulose, fatty acid glycerides, water, propylene glycol, macrogols, alcohol, crystalline cellulose, hydroxypropylcellulose, low-substituted hydroxypropylcellulose, carmelloses, povidone, polyvinyl alcohol, calcium stearate, etc. In this case, if necessary, a colorant, a stabilizer, an antioxidant, an antiseptic, a pH regulator, a tonicity agent, a solubilizing agent and/or a soothing agent, etc. can be added.

Granules, tablets or capsules can be coated with a coating base material, for example, such as hydroxypropylmethylcellulose, hydroxypropylmethylcellulose phthalate, etc.

These preparations can contain DHA and/or EPA in a proportion of 0.01% by mass or by weight or more, preferably 0.5 to 50% by mass or by weight.

The food products, to which the present invention can be applied, can be produced in a solid form, a semisolid from or a liquid form. The forms for formulation include various preparation forms such as tablets, pills, capsules, liquid formulations, syrups, powder, granules, etc.

The forms of the food products to which the present invention can be applied, include, for example, beverages (refreshing drink, tea drink, coffee drink, milk drink, fruit juice drink, carbonated drink, nutritional drink, powder drink, jelly drink, alcohol drink, etc.), breads, noodles, rice food products, jelly foods, confectionery (various snacks, baked confectionery, cakes, chocolate, gum, candy, tablet, etc.), soups, dairy products, frozen foods, fish processed products (fish meat sausage, kamaboko (boiled fish paste), chikuwa (fish sausage), hanpen (steamed cake of ground fish), etc.), livestock processed products (hamburger stakes, hams, sausages including Vienna sausage like a frankfurter, cheese, butter, yogurt, fresh cream, margarine, fermented milk etc.), instant foods, supplements, capsules, cereals, other processed foods; seasonings or materials thereof. These products may contain DHA and/or EPA in a proportion of 0.01% by mass or by weight, or more, preferably 0.1 to 5% by mass or by weight.

The organisms allowed to take the composition, the subjects to which the composition is administered and the subjects in need of treatment with DHA and/or EPA, according to the present invention, include human and various animals. The animals, for example, include dog, cat, mouse, rat, rabbit, cow, horse, monkey etc. The composition according to the present invention can be preferably applied, in particular, to pet animals, domestic animals and animals for livestock meats etc. with the problem of a neutral fat level increase.

A manual or a description including an explanation that the essential intake timing is the breakfast time and, if necessary, the intake timing is in the time range for intake within 6 hours after arousal from sleep including the breakfast time can be attached or added to the products, when the composition according to the present invention is provided to the users as the products such as various food products and pharmaceutical formulations, etc. Such explanation can be provided by enclosing it in a product package as an instruction pamphlet prepared separately from the product, or can be provided lo by printing it as instructions directly on the product per se or a product package (including an inner bag(s) for packaging divided products). The explanation can include information regarding the content of DHA and/or EPA in the product, the total intake or administration amount of DHA and/or EPA in the time for intake, or the continuous intake period etc. can be described. The product can be divided into the portions of a size to be taken within the time range for intake, and a necessary amount of the divided portions of the product can be packed in a product package.

### EXAMPLES

The present invention will be illustrated further in detail by examples below, but the present invention is not limited to the following examples.

### Example 1

### Lipid Metabolism Improving Effect by Intake in the Anterior Half of Active Phase of a Fish Oil Containing n-3 Unsaturated Fatty Acid Such as DHA.EPA, etc

### <Preparation of Mouse Feed>

As shown in Table 1, 4% by mass lard contained in a high fructose feed F2HFrD (manufactured by Oriental Yeast Co., Ltd.) was substituted by a fish oil containing n-3 unsaturated fatty acids such as DHA.EPA, etc., (DHA-22K, manufactured by Maruha Nichiro Corporation), to obtain a fish oil-containing modified F2HFrD feed. As the control, F2HFrD was used as a feed for mice.

TABLE 1
Compositions of Samples for Experiments

| | | Feed with 4% of |
|---|---|---|
| | Control Feed | Fish oil by mass |
| Component | (g/kg) | (g/kg) |
| Casein | 207 | 207 |
| Methionine | 3.0 | 3.0 |
| Fructose | 600 | 600 |
| Cellulose | 92.49 | 92.49 |
| AIN-93G mineral mixture | 35.0 | 35.0 |
| AIN-93 vitamin mixture | 10.0 | 10.0 |
| Choline hydrogen tartrate | 2.5 | 2.5 |
| Tert-butylhydroquinone | 0.010 | 0.010 |
| Lard | 50.0 | 10.0 |
| DHA-22K | — | 40.0 |

The numerical value for each component n Table 1 is the number of grams per kg of the feed.

AIN-93G mineral mixture in Table 1 has the following composition (gikg).
Calcium carbonate: 357
Potassium dihydrogen phosphate: 250
Tripotassium citrate: 28
Sodium chloride: 74
Potassium, sulfate: 46.6
Magnesium oxide: 24
Ferric citrate: 6.06
Smithsonite: 1.65
Manganese carbonate: 0.63
Cupric carbonate: 0.324
Potassium iodate: 0.01
Sodium selenate: 0.0103
Ammonium molybdate.4H₂O: 0.00795
Sodium metasilicate.9H₂O: 1.45
Potassium chromium sulfate.12H₂O: 0.275
Lithium chloride: 0.0174
Boric acid: 0.0815
Sodium fluoride: 0.0635
Nickel (ii) carbonate.4H₂O: 0.0306
Ammonium metavanadate: 0.0066
Sucrose: 209.7832

AIN-93 vitamin mixture in Table 1 has the following composition (g/kg).
Nicotinic acid: 3
Calcium pantothenate: 1.6
Pyridoxine hydrochloride: 0.7
Thiamine hydrochloride: 0.6
Riboflavin: 0.6
Folic acid: 0.2
D-biotin: 0.02
Vitamin B-12 (cyanocobalamine: 0.1%): 2.5
Vitamin E (all-rac-alpha-tocopherol acetate: 50%): 15
Vitamin A (all-trans-retinol palmitate: 500,000 U/g): 0.8
Vitamin D₃ (cholecalciferol: 400,000 U/g): 0.25
Vitamin K (phylloquinone): 0.075
Sucrose: 974.655

The fatty acid composition of DHA-22K is as described below.
Palmitic acid (16:0): 22.0%
Stearic acid (18:0): 5.7%
Oleic acid (18:1 n-9): 11.7%
Linoleic acid (18:2 n-6): 1.2%
Arachidonic acid (20:4 n-6): 1.8%
EPA (20:5 n-3): 5.1%
DHA (22:6 n-3): 27.3%
Others: 25.2%

### <Mouse Breeding and Time-Limited Feeding of Fish Oil Containing DHA.EPA>

ddY strain mice (5 week-old male, available from Japan SLC, Inc.) were bred for 3 weeks under the light-dark cycle consisting of 12 hours of the light period and 12 hours of the dark period (lighting at 0:00 o'clock, lights-out at 12:00 o'clock) (habituation rearing period).

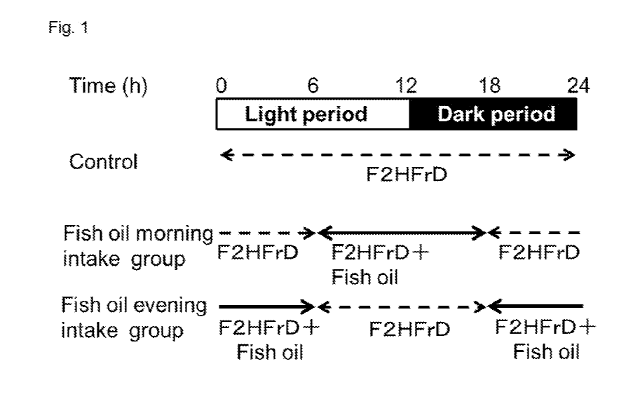

After the habituation rearing period, mice were divided into 3 groups (24 animals for each group). As shown in FIG. 1, the F2HFrD feed was given as a feed to the control group (CTRL) during the whole day for two weeks. The fish oil-containing modified F2HFrD feed (“F2HFrD+fish oil ” in FIG. 1) was given as a feed to the fish oil morning intake group (BF-FO) in 6:00 to 18:00 including the mouse activation beginning time, and the F2HFrD feed was given in the remaining time 18:00 to 6:00, for two weeks. The fish oil-containing modified F2HFrD feed was given as a feed to the fish oil evening intake group (DN-FO) in 18:00 to 6:00 including the mouse activation finishing time, and the F2HFrD feed was taken in the remaining time 6:00 to 18:00 for 2 weeks. The fish oil intake amount per day was 0.12±0.0084 g (average value±standard error) for the fish oil morning intake group, while 0.14±0.0089 g for the fish oil evening intake group. No statistically significant difference (t-test) was recognized.

### Test Example 1

### Effect on Blood Lipid Concentration

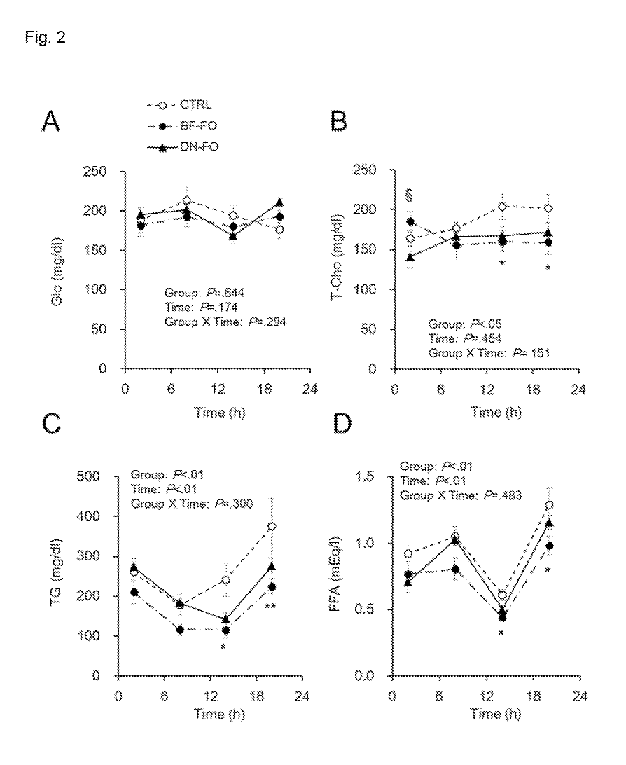

The time-limited feeding of the fish oil-containing modified F2HFrD feed was continued for 2 weeks, then, each four mice were killed for each group every 6 hours from 2:00 o'clock. The whole blood was collected from each mouse, and, then, plasma was separated. Each plasma was cryopreserved at minus 80 degree. FIG. 2 shows graphs showing the concentrations of glucose (Glu), free fatty acid (FFA), neutral fat (TG) and total cholesterol (T-Cho) in the plasma, which were measured by using the commercially available kits (LabAssay Glucose, LabAssay NEFA, LabAssay Triglyceride, LabAssay Cholesterol kit (manufactured by Wako Pure Chemical Industries, Ltd.), respectively. It was clarified that the plasma concentrations of the total cholesterol, the neutral fat and the free fatty acid in the fish oil morning intake group (BF-FO) were lowered statistically significantly as compared with the control group (CTRL). For the glucose concentration, no significant action was recognized.

### Test Example 2

### Effect on Amount of Lipid in Liver

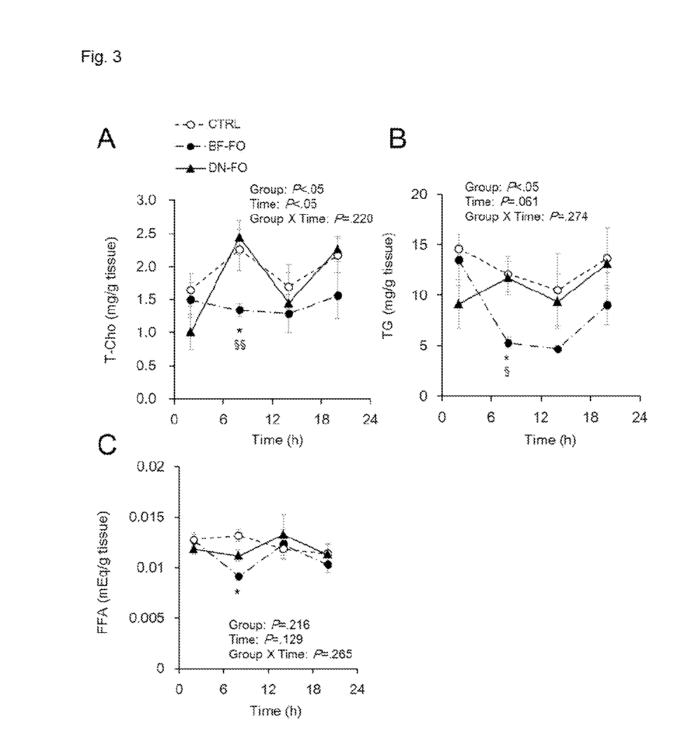

In the same manner as in Test Example 1, the time-limited feeding of the fish oil-containing modified F2HFrD feed was continued for 2 weeks, and, then, each four mice were killed for each group every 6 hours from 2:00 o'clock. A part of the liver was collected from each mouse and the lipid amount in the liver tissue was measured according to Non-Patent Document (Journal of Nutrition (2015), Vol. 145, No. 2, pp. 199-206, Oishi K. et al.). FIG. 3 shows graphs showing the contents of the free fatty acid (FFA), the neutral fat (TG) and the total cholesterol (T-Cho) per weight of the liver tissue. It was clarified that the contents of the total cholesterol, the neutral fat and the free fatty acid in the liver tissue in the fish oil morning intake group (BF-FO) were lowered statistically significantly as compared with the control group (CTRL). In the fish oil evening intake group (DN-FO). No significant difference was recognized as compared with the control group.

### Test Example 3

### Effect on Expression of mRNA of Fatty Acid Synthesis Genes in Liver Tissue

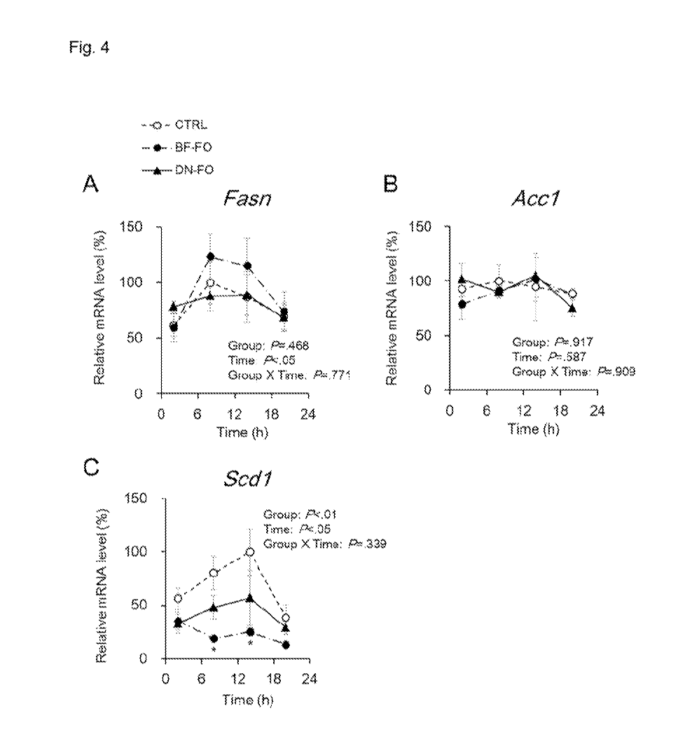

In the same manner as in Test Example 1, the time-limited feeding of the fish oil-containing modified F2HFrD feed was continued for 2 weeks, and, then, each four mice were killed for each group every 6 hours from 2:00 o'clock. A part of white fat was collected from each mouse, the whole mRNA was extracted, and, then, the expression amounts of the fatty acid synthesis related genes (Fasn, Acc1, Scd1) were measured by a quantitative PCR method. FIG. 4 shows graphs showing the analysis results of the expression amounts of the fatty acid synthesis related genes (Fasn, Acc1, Scd1). In FIG. 4, the expression amount of mRNA is shown as a ratio when the peak value in the control group (CTRL) is 100%. It was clarified that the mRNA expression amount of the Scdl gene in the fish oil morning intake group (BF-FO) was lowered statistically significantly as compared with the control group. In the fish oil evening intake group (DN-FO), no significant difference was recognized as compared with the control group (CTRL).

### Test Example 4

### Effect on Blood Fatty Acid Concentration

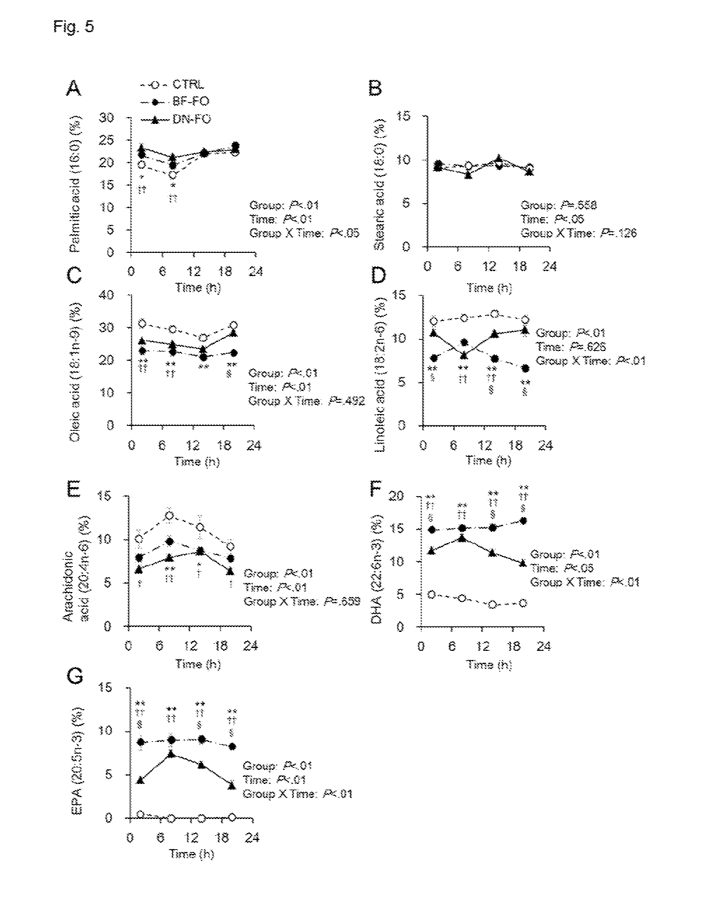

FIG. 5 shows graphs of the fatty acid concentration of each plasma in the same manner in Test Example 1, which was obtained by killing mice every 6 hours from 2:00 o'clock after the fish oil time-limited feeding for 2 weeks, and separating the plasm from blood of each mouse. The fatty acid was extracted from the plasma (100 μL) with a chloroform-methanol solution according to a method of Canadian Journal of Biochemistry and Physiology (1959), Vol. 37, No. 8, pp. 911-917, E. G. Bligh, W. J. Dyer. The Methyl esterification method (boron trifluoride-methanol method) (“Standard methods for the analysis of fats, oils and related materials”, established by Japan Oil Chemists' Society, 2.4.1.2-2013) was partially modified and the fatty acid concentration measurement was carried out by the partially modified method. A 0.5 N sodium hydroxide methanol solution of 1.5 mL was added to the extracted sample and the sample mixture thus obtained was heated at 100° C. for 9 minutes. After cooling, 2 mL of a methanol solution of boron trifluoride methanol complex was added to the sample mixture, and the resultant mixture was heated at 100° C. for 7 minutes. After cooling again, 3 mL of hexane was added to the mixture and is the mixture was stirred. Further. 3 mL of distilled water was added to the mixture and the mixture was stirred, allowed to stand still to cause separation into two layers, and, then, the upper layer was collected. Water was removed from the upper layer over anhydrous sodium sulfate, and, then, the upper layer was subjected to gas chromatography. In the fish oil morning intake group, the concentrations of DHA and EPA which were contained in the fish oil, which are scarcely synthesized in a body, were statistically significantly higher throughout the day as compared with the fish oil evening intake group. No difference in the concentration of palmitic acid contained in the amount similar to DHA in the fish oil.

When the absolute amounts of fatty acids were measured (Table 2), the amounts of DHA and EPA were significantly higher in the fish oil morning intake group (BF-FO) at 20:00 o'clock as compared with the fish oil evening intake group (DN-FO). It is known that n-3 unsaturated fatty acids such as DHA, etc. transfer into the systemic circulation via lymph vessels and the plasma concentrations thereof reach the maximum value 9 hours after administration (Non-Patent Document 2). Therefore, it was considered that n-3 unsaturated fatty acids taken at the breakfast time (6:00 to 18:00) were transferred into the systemic circulation. In contrast, in the fish oil evening intake group (DN-FO), no significant difference at 8:00 o'clock was observed as compared with the fish oil morning intake group (BF-FO). These results suggested that the plasma n-3 unsaturated fatty acid concentration was highly increased in the fish oil morning intake group (BF-FO).

### [Table 2]

TABLE 2

| nmol/ | | | | | | |
|---|---|---|---|---|---|---|
| sample | 2:00 | | | 8:00 | | |
| mg | control | BF-FO | DN-FO | control | BF-FO | DN-FO |
| 16:0 | 3.6 ± 0.7 | 3.8 ± 0.6 | 4.1 ± 1.1 | 3.0 ± 0.7 | 2.3 ± 0.4 | 3.3 ± 0.6* |
| 18:0 | 1.5 ± 0.2 | 1.5 ± 0.2 | 1.5 ± 0.4 | 1.4 ± 0.2 | 1.0 ± 0.2 | 1.2 ± 0.2 |
| 18:1 n-9 | 5.2 ± 1.1 | 3.7 ± 1.0 | 4.2 ± 0.9 | 4.6 ± 0.9 | 2.4 ± 0.4 | 3.5 ± 0.8* |
| 18:2 n-6 | 2.0 ± 0.2 | 1.3 ± 0.2 | 1.7 ± 0.4* | 2.0 ± 0.3 | 1.1 ± 0.3 | 1.2 ± 0.2 |
| 20:4 n-6 | 1.5 ± 0.4 | 1.2 ± 0.2 | 1.0 ± 0.3 | 1.8 ± 0.3 | 1.0 ± 0.3 | 1.0 ± 0.2 |
| 22:6 n-3 | 0.7 ± 0.1 | 2.0 ± 0.3 | 1.6 ± 0.4 | 0.6 ± 0.1 | 1.4 ± 0.3 | 1.7 ± 0.2 |
| 20:5 n-3 | 0.1 ± 0.1 | 1.3 ± 0.3 | 0.7 ± 0.2** | 0.0 ± 0.0 | 0.9 ± 0.3 | 1.0 ± 0.2 |
| nmol/ | | | | | | |
| sample | 14:00 | | | 20:00 | | |
| mg | control | BF-FO | DN-FO | control | BF-FO | DN-FO |
| 16:0 | 4.5 ± 1.5 | 2.8 ± 0.4 | 3.1 ± 0.3 | 5.2 ± 1.7 | 3.7 ± 0.5 | 4.7 ± 0.5 |
| 18:0 | 1.8 ± 0.5 | 1.1 ± 0.2 | 1.3 ± 0.1 | 1.9 ± 0.6 | 1.3 ± 0.2 | 1.4 ± 0.5 |
| 18:1 n-9 | 5.0 ± 1.6 | 2.4 ± 0.4 | 3.0 ± 0.5 | 6.7 ± 2.6 | 3.2 ± 0.5 | 4.7 ± 1.8 |
| 18:2 n-6 | 2.4 ± 0.6 | 0.9 ± 0.1 | 1.3 ± 0.1 | 2.6 ± 0.5 | 0.9 ± 0.1 | 1.8 ± 0.7 |
| 20:4 n-6 | 1.9 ± 0.5 | 0.9 ± 0.1 | 1.0 ± 0.1 | 1.7 ± 0.2 | 1.0 ± 0.1 | 1.3 ± 0.4 |
| 22:6 n-3 | 0.5 ± 0.1 | 1.5 ± 0.3 | 1.2 ± 0.2 | 0.7 ± 0.2 | 2.0 ± 0.3 | 1.5 ± 0.4* |
| 22:5 n-3 | 0.0 ± 0.0 | 1.0 ± 0.1 | 0.7 ± 0.1** | 0.0 ± 0.0 | 1.1 ± 0.2 | 0.6 ± 0.3** |

BF-FO vs DN-FO, Tukey-kramer,
*p < 0.05,
**p < 0.01

### Example 2

### Influence to Discharge Amount of Fatty Acid into Feces by Difference of Intake Time of Fish Oil Containing n-3 Unsaturated Fatty Acid Such as DHA-EPA, etc

### <Preparation of Mouse Feed >

The same feed as in Example 1 was used.

### <Mouse Breeding and Time-Limited Feeding of Fish Oil Containing DHA.EPA>

The same time-limited feeding as in Example 1 was conducted.

The time-limited feeding of the fish oil-containing modified F2HFrD feed was continued for 9 days, and, then, the total feces of each mouse for one day was collected. The amounts of fatty acids in the feces were measured. The fatty acids were extracted from the feces in the same manner as in Example 1. Test Example 4 and subjected to methyl esterification. The samples thus obtained were annualized by gas chromatography. The lipid amounts in the one days feces are shown in Table 3. The tendency of the smaller amounts in the fish oil morning intake group (BF-FO) as compared with the fish oil evening intake group (DN-FO) was observed. It was suggested that lipid was more incorporated. Particularly, in the fish oil morning intake group (BF-FO), the discharge amount of n-3 unsaturated fatty acids into the feces was smaller, and also the n-3/n-6 ratio was lower as compared with the fish oil evening intake group (DN-FO). It was considered that, when the fish oil was taken as the breakfast, a larger amount of n-3PUFA was incorporated into blood, and, thus, the plasma n-3 unsaturated fatty acid concentration increased.

TABLE 3

| | Control (n = 6) | | BF-DHA (n = 4) | | DF-DHA (n = 4) | | |
|---|---|---|---|---|---|---|---|
| mg/day | mean | SD | mean | SD | mean | SD | |
| SFA | 10.57 | 5.11 | 5.55 | 2.13 | 10.74 | 2.75 | † |
| MUFA | 1.97 | 0.83 | 0.93 | 0.31 | 2.02 | 0.59 | * |
| n-6 unsaturated | 0.24 | 0.11 | 0.12 | 0.03 | 0.19 | 0.05 | |
| fatty acid | | | | | | | |
| n-3 unsaturated | 0.00 | 0.00 | 0.05 | 0.04 | 0.14 | 0.05 | * |
| fatty acid | | | | | | | |
| Others | 1.68 | 0.71 | 1.23 | 0.35 | 2.03 | 0.60 | |
| (unidentified) | | | | | | | † |
| Total fatty acid | 14.46 | 6.68 | 7.88 | 2.84 | 15.11 | 3.95 | † |
| n-3/n-6 | 0.00 | 0.00 | 0.38 | 0.24 | 0.71 | 0.16 | * |

BF-FO vs DN-FO, LSD,
*p < 0.05,
†p < 0.1

### Example 3

### Influence to Incorporation Amount of Fatty Acid into Blood by Difference of Intake Time of Fish Oil Containing n-3 Unsaturated Fatty Acid Such as DHA.EPA, etc. Number 1

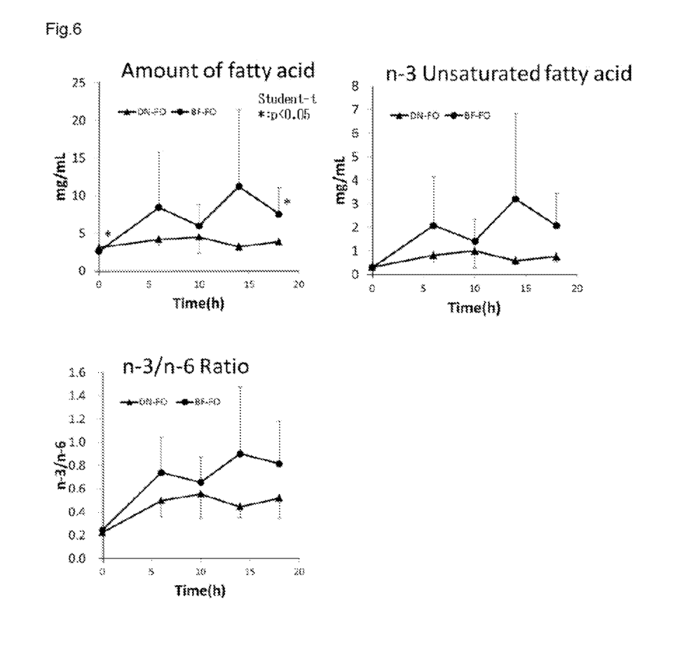

ddY strain mice (7 week-old male, available from Japan SLC, Inc.) were divided into the fish oil evening intake group (ON-FO) with administration of the fish oil (10 mg/kg) in single dose at 1:00 o'clock and the fish oil morning intake group (BF-FO) with administration of the fish oil in single dose at 13:00 o'clock (25 animals for each group). The fish oil was administered after fasting of 5 hours for each mouse. In each group, each 5 mice were killed at 0, 6, 10, 14 and 18 hours after administration, and plasma was collected for each mouse. The fatty acid was extracted from each plasm in the same manner as in Example 1, Test Example 4, and subjected to methyl esterification. Each sample was annualized by gas chromatography. FIG. 6 shows graphs showing the fatty acid amounts. the n-3 unsaturated fatty acid amounts and the n-3/n-6 ratio measured in each plasma of each group. In the fish oil morning intake group (BF-FO), the incorporation amounts of fatty acids into blood were large corresponding to the result of small discharge amount into feces of Example 2.

### Example 4

### Influence to Incorporation Amount of Fatty Acid into Blood by Difference of Intake Time of Fish Oil Containing n-3 Unsaturated Fatty Acid Such as DHA.EPA etc. Number 2

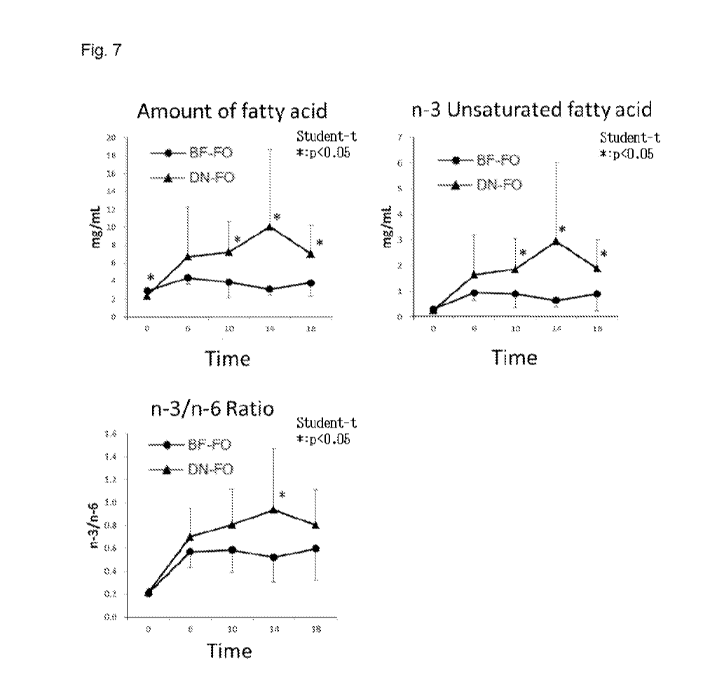

The same test as in Example 3 was carried out while the sample number was increased. 7 week-old ddy mice (98 mice) were divided into the fish oil evening intake group (DN-FO) and the fish oil morning intake group (BF-FO) (each 49 animals). The fish oil was administered to each mouse after fasting of 5 hours. In each group, each 9 mice were killed at 0, 6. 10, 14 and 18 hours after administration (8 mice only at 0 hour), and plasma was collected from each mouse. FIG. 7 shows graphs showing the plasma fatty acid amounts, the n-3 unsaturated fatty acid amounts and the n-3/n-6 ratios measured in each plasma of each group. Also in the case of an increase of the sample number, the amount of incorporation of fatty acids into blood was larger in the fish oil morning intake group (BF-FO) as the same manner as Example 3.

### Example 5

### Influence Exerted on Blood Lipid in Healthy Person by Difference of Intake Time of Fish Oil Containing n-3 Unsaturated Fatty Acid Such as DHA.EPA, etc

In this test, a fish oil-added fish meat sausage containing 850 mg of DHA and 200 mg of EPA was used, while a placebo fish meat sausage in which olive oil was blended instead of the fish oil was used.

Twenty 20 old or more and 60 old or less healthy Japanese men and women were divided into two groups, i. e., each composed of 10 members. The test was conducted for 8 weeks. The fish oil morning intake group was allowed to take in one fish oil-added fish meat sausage at the breakfast and to take in one placebo fish meat sausage in the supper. The fish oil evening intake group was allowed to take one placebo fish meat sausage at the breakfast and to take one fish oil-added fish meat sausage in the supper. The breakfast was taken within 6 hours after arousal from sleep for each group.

Each blood sample was taken by the morning blood sampling at the standard time (0 h) at 0, 4 and 8 weeks from the beginning of the test, and by the evening blood sampling, at (8 h), i. e., 8 hours after the standard time (0 h) for the morning blood sampling. A prescribed diet was taken until 8 hours before the morning blood sampling, and fasting was continued until completion of the morning blood sampling. After completion of the morning blood sampling, a prescribed diet and a test diet were taken, and, thereafter, fasting was continued for 8 hours or more until the evening blood sampling. The blood neutral fat amounts and the blood fatty acid amounts were measured for each blood sample and evaluated.

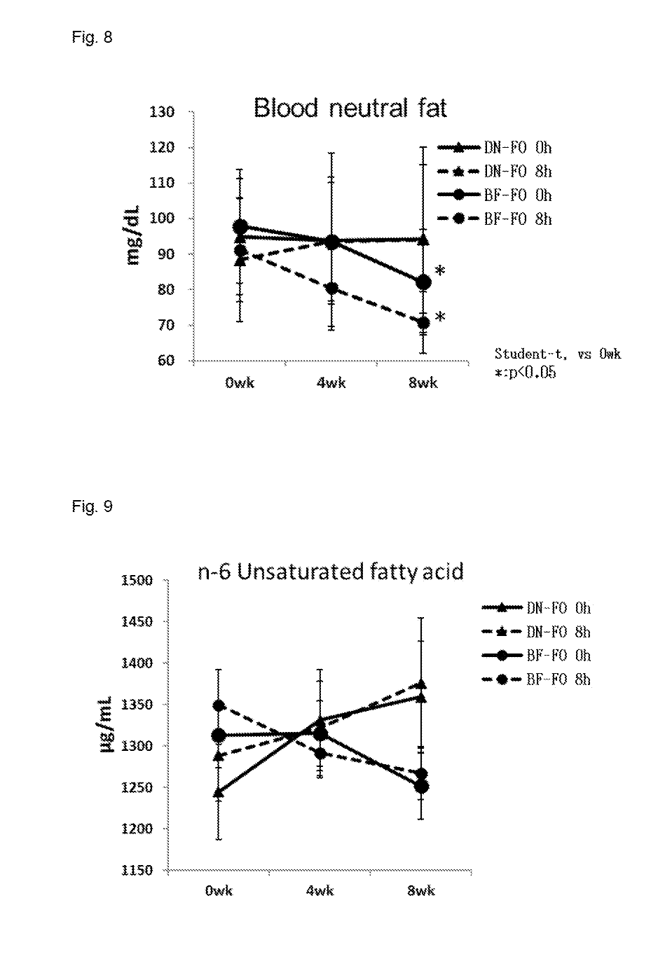

FIG. 8 shows transition of the blood neutral fat. In the fish oil morning intake group, the blood neutral fat at 8 weeks was lowered significantly in the blood sampling both at 0 h and 8 h as compared with that before intake.

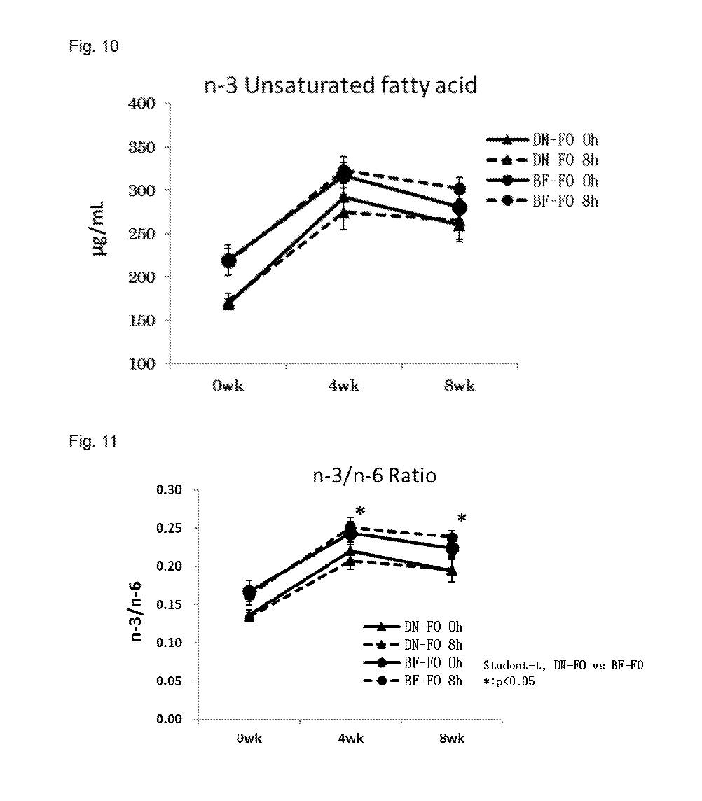

FIGS. 9, 10 and 11 shows the values of n-6 unsaturated fatty acids, n-3 unsaturated fatty acids and the n-3/n-6 ratio, for each group, respectively. In the fish oil morning intake group, the n-6 unsaturated fatty acid was lowered as compared with that before intake in the blood samplings both at 0 h and 8 h at 8 weeks, while in the fish oil evening intake group, the n-6 unsaturated fatty acid increased as compared with that before intake. It was considered that neutral fat was lowered by enhancing the n-3/n-6 ratio.

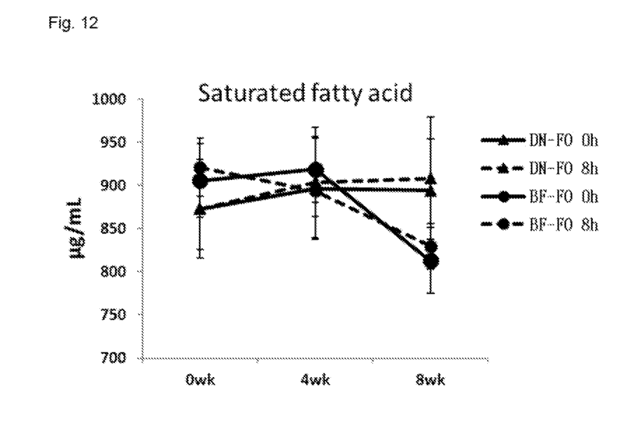

FIG. 12 shows the values of saturated fatty acids. Since the saturated fatty acid was lowered as compared with that before intake in the blood samplings both at 0 h and 8 h at 8 weeks in the fish oil morning intake group, a possibility that beta-oxidation increased to suppress resynthesis of neutral fat was also supposed.

These results show the usability of the intake of a fish oil as the breakfast, not only for the mice but also for human.

What is claimed is:

1. Use of a composition for decreasing or suppressing an increase in the neutral fat level of a subject in need of intake of the composition,
wherein the composition contains docosahexaenoic acid (DHA) and/or eicosapentaenoic acid (EPA) as an active ingredient: and
wherein the composition is taken by the subject in a time range for intake, essentially including a breakfast time and within 6 hours after arousal from sleep including the breakfast time.

2. The use according to claim 1, where the composition is taken by the subject only in the time range.

3. The use according to claim 1, wherein the composition contains docosahexaenoic acid and/or eicosapentaenoic acid in an amount of 0.01% by mass or more.

4. The use according to claim 1, wherein the composition contains docosahexaenoic acid and/or eicosapentaenoic acid in an amount at which the total intake dose of docosahexaenoic acid and/or eicosapentaenoic acid in said time for intake is selected from the range of 500 mg to 2000 mg.

5. The use according to claim 1, wherein the composition contains a fish oil containing docosahexaenoic acid and eicosapentaenoic acid.

6. The use according to claim 1, wherein the composition is a pharmaceutical formulation.

7. The use according to claim 1, wherein the composition is a food product.

8. The use according to claim 7, wherein the food product is a functional food product.

9. The use according to claim 7, wherein the food product is a food product for breakfast.

10. A method of treating a subject in need of treatment for decreasing or suppressing an increase in the neutral fat level comprising:
providing the subject a composition for the treatment,
wherein the composition contains docosahexaenoic acid (DHA) and/or eicosapentaenoic acid (EPA) as an active ingredient; and
wherein the composition is taken by the subject in a time range for intake, essentially including a breakfast time and within 6 hours after arousal from sleep including the breakfast time.

11. The method according to claim 10, where the composition is taken by the subject only in the time range.

12. The method according to claim 10, wherein the composition contains docosahexaenoic acid and/or eicosapentaenoic acid in an amount of 0.01% by mass or more.

13. The method according to claim 10, wherein the composition contains docosahexaenoic acid and/or eicosapentaenoic acid in an amount at which the total intake dose of docosahexaenoic acid and/or eicosapentaenoic acid in said time for intake is selected from the range of 500 mg to 2000 mg.

14. The method according to claim 10, wherein the composition contains a fish oil containing docosahexaenoic acid and eicosapentaenoic acid.

15. The method according to claim 10, wherein the composition is a pharmaceutical formulation.

16. The method according to claim 10, wherein the composition is a food product.

17. The method according to claim 16, wherein the food product is a functional food product.

18. The method according to claim 16, wherein the food product is a food product for breakfast.

19. A method of using an active ingredient for decreasing or suppressing an increase in the neutral fat level, in production of a composition for decreasing or suppressing an increase in the neutral fat level,
wherein the active ingredient comprises docosahexaenoic acid (DHA) and/or eicosapentaenoic acid; and
wherein the composition is provided for intake by a subject in a time range for intake, essentially including a breakfast time and within 6 hours after arousal from sleep including the breakfast time.

20. The method according to claim 19, where the composition is taken by the subject only in the time range.

21. The method according to claim 19, wherein the composition contains docosahexaenoic acid and/or eicosapentaenoic acid in an amount of 0.01% by mass or more.

22. The method according to claim 19, wherein the composition contains docosahexaenoic acid and/or eicosapentaenoic acid in an amount at which the total intake dose of docosahexaenoic acid and/or eicosapentaenoic acid in said time for intake is selected from the range of 500 mg to 2000 mg.

23. The method according to claim 19, wherein the composition contains a fish oil containing docosahexaenoic acid and eicosapentaenoic acid.

24. The method according to claim 19, wherein the composition is a pharmaceutical formulation.

25. The method according to claim 19, wherein the composition is a food product.

26. The method according to claim 25, wherein the food product is a functional food product.

27. The method according to claim 25, wherein the food product is a food product for breakfast.
